(19) United States
(12) Patent Application Publication
(10) Pub. No.: US 2021/0311003 A1
(43) Pub. Date: Oct. 7, 2021

(54) Carbon Dioxide Capture Device and Control Interface

(71) Applicant: Earthly Labs Inc., Austin, TX (US)

(72) Inventor: Amy George, Austin, TX (US)

(73) Assignee: Earthly Labs Inc., Austin, TX (US)

(21) Appl. No.: 16/696,431

(22) Filed: Nov. 26, 2019

Related U.S. Application Data
(60) Provisional application No. 62/771,725, filed on Nov. 27, 2018.

(51) Int. Cl. G01N 33/00

(52) U.S. Cl. CPC G01N 33/004; G01N 2033/0068; G01N 33/0062; G01N 33/0073

(57) ABSTRACT

In some embodiments, a carbon capture system may include a carbon capture device including a plurality of sensors, circuitry, and various controllable elements that may process a waste gas stream to produce a purified, liquid CO₂ gas product. The sensors may be distributed through various stages in the processing of the waste gas stream, and each sensor may produce a sensor signal proportional to a parameter to be sensed and may provide the sensor signal to a control circuit.

### CROSS-REFERENCE TO RELATED APPLICATION(S)

This application is a non-provisional of and claims priority to U.S. Provisional Patent Application No. 62/771,725 filed on Nov. 27, 2018 and entitled “Interface Configured to Monitor and Control a Carbon Dioxide Capture Device”, which is incorporated herein by reference in its entirety.

### FIELD

The present disclosure is generally related to systems and devices configured to capture carbon dioxide, and more particularly, to an interface configured to monitor and control a carbon dioxide capture device configured to produce purified carbon dioxide for reuse.

### BACKGROUND

Climate change is one of the biggest challenges that our planet has faced. The NASA's (National Aeronautics and Space Administration) early release of satellite images and measurement data depicted rapid changes in climate and temperature around the globe that demonstrate unprecedented warming trends. The 2015 Paris Agreement signed by 192 countries defined a global plan of action to slow climate change. One aspect of the 2015 Paris Agreement involves reduction in carbon dioxide (CO₂) emissions.

Beer (an aqueous solution of 3-10% ethanol by volume) is produced by the anaerobic fermentation of glucose by the yeast Saccharomyces cerevisiae, which produces a byproduct of two molecules of ethanol and two molecules of CO₂ for each molecule of glucose. Since beer consumers typically enjoy carbonation, the CO₂ is not entirely undesirable. However, carbonation of beer does not utilize all the CO₂ produced during fermentation.

Conventionally, the CO₂ byproduct may be vented in large quantities, either into the brew-making premises or directly into the atmosphere, by craft beer breweries. In some instances, the venting of carbon dioxide has raised environmental issues that could cause state regulators, federal regulators, or both to take action to fine, shutdown, or regulate their operations.

### SUMMARY

Embodiments of systems, devices, and methods are described below that may be configured to recover CO₂ from a waste stream and repurpose the recovered CO₂ byproduct for other applications. In some implementations, the recovered CO₂ byproduct may be used for carbonation of the final beverage product, whether in bottles, cans, or kegs, purging of process lines and vessels to exclude air, and dispensing beverages in an on-site tasting room or brewpub. In some implementations, the recovered CO₂ byproduct may be repurposed to refill tanks with beverage-grade CO₂, which may be sold to third-parties for reuse with other products, such as soft drinks, carbonated water, and so on.

In some implementations, the systems, methods, and devices described below may be configured to capture CO₂ from a waste stream of a process and to process the captured CO₂ to produce a food-grade CO₂ that may be stored in a tank. In some implementations, the systems, methods, and devices may be configured to operate in conjunction with intermittent, low concentration CO₂ sources.

In some implementations, a system may be configured to host a CO₂ recycling ecosystem of CO₂ production devices and CO₂ purchasers. The system may include an interface and back-end system configured to enable sales of recovered CO₂ to third-parties. In some implementations, the system may also forecast production, forecast consumption, and coordinate inventories and sales, as well as CO₂ pick-up and delivery.

In some implementations, systems, devices, and methods may be configured to monitor and control remote field equipment (such as sensors, actuators, blowers, refrigeration units, heaters, and the like), automatically. The systems, devices, and methods may be configured to remotely monitoring and control CO₂ capture devices and associated field equipment through a communications network. The CO₂ capture devices may be used in connection with fermentation processes (food and beverage processes, CO₂ waste streams, other processes that may generate CO₂ as a byproduct, or any combination thereof). The CO₂ capture devices may receive control signals from the control circuitry and may adjust one or more parameters in response to the control signals to facilitate waste gas processing and CO₂ gas capture and purification.

In some implementations, a CO₂ capture system may include a plurality of sensors, circuitry and various actuatable components that may be controlled to process a waste gas stream to capture CO₂ from a waste stream and to produce a purified, liquid CO₂ product, which may be food-grade level purity. The sensors may be distributed across various stages in the processing of the CO₂ waste gas stream. Each sensor may produce a sensor signal proportional to a parameter to be sensed and may provide the sensor signal to a control circuit.

In some implementations, the control circuit may be configured to receive one or more sensor signals to automatically measure and monitor one or more parameters of the CO₂ capture process. The control circuit may analyze the one or more parameters and may control various components to manage the CO₂ capture process. The control circuit may communicate data related to the control operations and the sensed parameters to a computing device through a network. In some instances, the sensed parameters may include information related to the volume of captured CO₂. The circuit may sometimes receive control signals from the computing device and may act on the control signals to control the components. The computing device may provide a graphical interface including information derived from the data. The information may include first data corresponding to one or more parameters of the CO₂ capture device, second data corresponding to production of CO₂ by the CO₂ capture device. Other embodiments are also possible.

In some implementations, the control circuit may receive sensor signals from a plurality of sensors and may monitor the various components of the CO₂ capture process based on the sensor signals. The control circuit may send data related to the sensor signals through a communications network to a computing device, which may process the received data to determine control signals to manage operation of the CO₂ capture device. Other embodiments are also possible.

In some implementations, a system may include hardware, software, and sensors that may cooperate to remotely monitor, analyze, measure, and control a CO₂ capture process. The hardware may include a piece of remote field equipment (a CO₂ capture device) for separating the CO₂ gas from a waste gas stream and a CO₂ storage device (e.g., one or more CO₂ canisters or tanks). The software may execute on a processing circuit of the CO₂ capture device or on a computing device in communication with the CO₂ capture device through a network. The software may be configured to receive a set of chemical process input parameters (e.g., sensor data) and a set of desired chemical process output parameters and may be configured to generate control signals to control one or more parameters of the CO₂ capture device to achieve the desired chemical process output parameters. Additionally, the software may cooperate with the circuit to remotely monitor and analyze data from the circuit to provide data analytics. In some embodiments, the software may include an open application programming interface (API) that may allow customers to import data to website to track the CO₂ capture impact in real-time. Other embodiments are also possible.

In some implementations, a computing device may include a display, a network interface, and a processor coupled to the network interface and the display. The network interface may be configured to receive a signal through a network from a CO₂ capture device configured to separate CO₂ from a mixed gas waste stream that includes CO₂. The processor may be configured to provide a graphical interface to the display. The graphical interface may include data related to the CO₂ capture process and may include one or more controls accessible by a user to interact with one or more of a parameter and a visualization of the CO₂ capture process.

### BRIEF DESCRIPTION OF THE DRAWINGS

The detailed description is set forth with reference to the accompanying figures. In the figures, the left most digit(s) of a reference number identifies the figure in which the reference number first appears. The use of the same reference numbers in different figures indicates similar or identical items or features.

FIG. 1 depicts a block diagram of a system including an interface configured to monitor and control a CO₂ capture device, in accordance with certain embodiments of the present disclosure.

FIG. 2 depicts a block diagram of a system including a local server configured to provide an interface to monitor and control a CO₂ capture device, in accordance with certain embodiments of the present disclosure.

FIG. 3 depicts a block diagram of a system including a control and maintenance server configured to provide an interface to monitor and control a CO₂ capture device, in accordance with certain embodiments of the present disclosure.

FIG. 4 depicts a diagram of components of a system including multiple interfaces for monitoring and controlling a CO₂ capture device, in accordance with certain embodiments of the present disclosure.

FIG. 5 depicts a graphical interface of a CO₂ capture device including collected data associated with a CO₂ capture device, in accordance with certain embodiments of the present disclosure.

FIG. 6 depicts a graphical interface of a CO₂ capture device including a graphical representation of the CO₂ capture device, in accordance with certain embodiments of the present disclosure.

FIG. 7 depicts a block diagram of a CO₂ capture device including a plurality of sensors and controllable components, in accordance with certain embodiments of the present disclosure.

FIG. 8 depicts a device including a graphical interface accessible by an operator to view data associated with a CO₂ capture device, in accordance with certain embodiments of the present disclosure.

FIG. 9 depicts a graphical interface accessible by an operator to view data associated with a CO₂ capture device, in accordance with certain embodiments of the present disclosure.

FIG. 10 depicts a graphical interface accessible by an operator to view data associated with a CO₂ capture device, in accordance with certain embodiments of the present disclosure.

FIG. 11 depicts a graphical interface accessible by an operator to view data associated with a CO₂ capture device, in accordance with certain embodiments of the present disclosure.

FIG. 12 depicts a graphical interface accessible by an operator to view data associated with a CO₂ capture device, in accordance with certain embodiments of the present disclosure.

FIG. 13 depicts a graphical interface accessible by an operator to view map data associated with a CO₂ capture device, in accordance with certain embodiments of the present disclosure.

FIG. 14 depicts a graphical interface accessible by an operator to view data associated with a CO₂ capture device, in accordance with certain embodiments of the present disclosure.

While implementations are described in this disclosure by way of example, those skilled in the art will recognize that the implementations are not limited to the examples or figures described. It should be understood that the figures and detailed description thereto are not intended to limit implementations to the particular form disclosed, but on the contrary, the intention is to cover all modifications, equivalents, and alternatives falling within the spirit and scope as defined by the appended claims. The headings used in this disclosure are for organizational purposes only and are not meant to limit the scope of the description or the claims. As used throughout this application, the work “may” is used in a permissive sense (in other words, the term “may” is intended to mean “having the potential to”) instead of in a mandatory sense (as in “must”). Similarly, the terms “include”, “including”, and “includes” mean “including, but not limited to”.

### DETAILED DESCRIPTION OF ILLUSTRATIVE EMBODIMENTS

Embodiments of systems, devices, and methods are described below that may be configured to recover CO₂ from a waste stream and to repurpose the recovered CO₂ byproduct for other applications. In some implementations, the recovered CO₂ byproduct may be used for carbonation of the final beverage product, whether in bottles, cans, or kegs, purging of process lines and vessels to exclude air, and dispensing beverages in an on-site tasting room or brewpub. In some implementations, the recovered CO₂ byproduct may be repurposed to refill tanks with beverage-grade CO₂, which may be sold to third-parties for reuse with other products, such as soft drinks, carbonated water, and so on.

Embodiments of systems, methods, and devices are described below that may be configured to provide a graphical interface to a display. In a first embodiment, a CO₂ capture device may include a plurality of sensors, a processor, and a display (such as a touchscreen display) on which a graphical interface may be displayed. The graphical interface may include first information about the CO₂ capture device, such as sensor data; second data related to production of purified CO₂ gas, such as CO₂ concentration, purity levels, volume, and so on; or any combination thereof. Further, the graphical interface may include one or more control options accessible by an operator to select between visualizations of the first data, the second data, or both; to selectively adjust one or more parameters associated with the CO₂ capture device; or any combination thereof.

In some embodiments, the CO₂ capture device may be configured to communicate data related to the sensor data, the production of the purified CO₂ gas, other data, or any combination thereof to a computing device, such as a computer server, through a network. The server may be a local server within the same enterprise as the CO₂ capture device, a control and maintenance server, or any combination thereof. In some examples, the CO₂ capture device may communicate data related to the production of the purified CO₂ gas to one or more portable computing devices, such as smartphones or other computing devices associated with customers of the enterprise. In some implementations, the data may include instructions to render a graphical interface.

In other implementations, the data may be presented within a graphical interface of an application executing on a computing device, a smartphone, another device, or any combination thereof. The graphical interface may be generated by an Internet browser application, by another application, or any combination thereof. In some implementations, the graphical interface may provide information about the amount of CO₂ gas that has been captured by the CO₂ capture device, information about the environmental impact of the captured CO₂ gas, other information, or any combination thereof.

In an example, the CO₂ capture device may be configured to process a waste gas stream comprised of a mixed gas including CO₂ from a source, such as an alcohol fermentation tank or another mixed gas waste stream. The CO₂ capture device may include a plurality of sensors and may be configured to monitor a plurality of parameters associated with the waste gas stream, components of the CO₂ capture device, and the purified CO₂ gas. The CO₂ capture device may provide a graphical interface to a display of the CO₂ capture device. The graphical interface may include data related to the plurality of parameters and one or more selectable controls accessible by an operator to control operation of the CO₂ capture device.

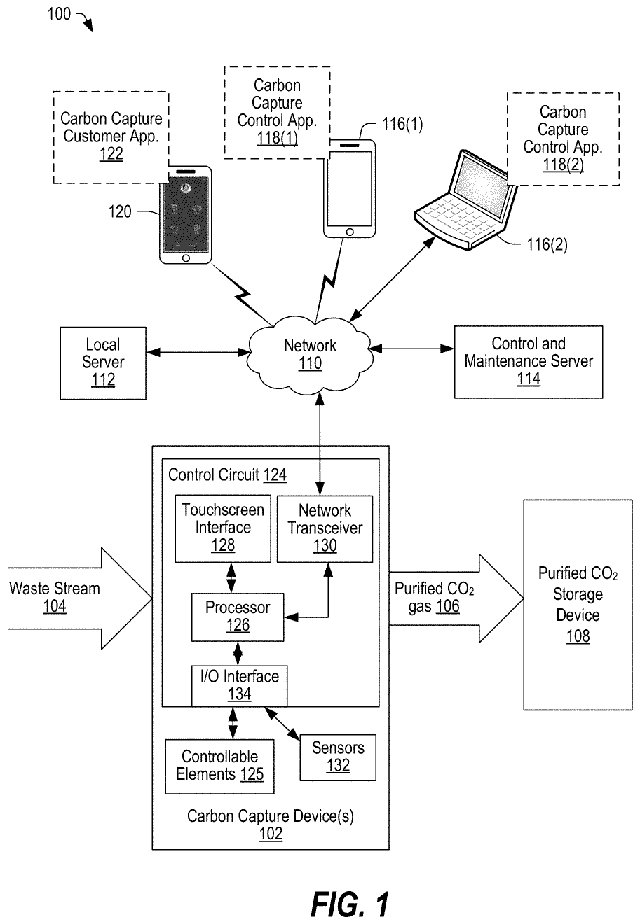

The CO₂ capture devices, the local server, the control and maintenance server, the computing devices (laptops, tablet computers, smartphones, or any combination thereof), and the corresponding applications and circuitry may cooperate to provide monitoring functionality, control functionality, or any combination thereof with respect to the capturing of purified CO₂ gas from a waste stream comprised of mixed gas. An implementation of a system configured to provide monitoring and control of one or more CO₂ capture devices is described below with respect to FIG. 1.

FIG. 1 depicts a block diagram of a system 100 including an interface configured to monitor and control a CO₂ capture device 102, in accordance with certain embodiments of the present disclosure. The CO₂ capture device 102 may include an input configured to receive a waste stream 104 comprised of a mixture of gases, including water, oxygen, CO₂, other gases, particles, other contaminants, or any combination thereof. The CO₂ capture device 102 may include an output configured to provide purified CO₂ gas 106 to a purified CO₂ storage device 108.

The CO₂ capture device 102 may be configured to communicate with a local server 112, a control and maintenance server 114, and one or more computing devices, such as computing devices 116 and 120, through a network 110. The network 110 may include a local area network, a public switched telephone network, a satellite network, a cellular network, the Internet, or any combination thereof. In the illustrated example, the computing devices 120 and 116(1) are depicted as smartphones, and the computing device 116(2) is depicted as a laptop computer. However, other computing devices are also possible, including tablet computers, desktop computers, or other data processing devices capable of communicating with the CO₂ capture device 102 through the network 110.

The local server 112 may include a computer server device associated with and controlled by an enterprise associated with the installed CO₂ capture device 102. For example, the local server 112 may be a computing device within a microbrewery or other facility that includes a source of the waste stream, such as a fermentation tank.

The control and maintenance server 114 may be a computer server device that is controlled by a third-party enterprise or controlled by a headquarters of the company in which the CO₂ capture device 102 is installed. For example, if the CO₂ capture device 102 is installed in a microbrewery that is part of a larger chain of restaurants, the control and maintenance server 114 may be associated with a company headquarters or other facility. Alternatively, the control and maintenance server 114 may be controlled by an installer of the CO₂ capture device 102. Other implementations are also possible.

The computing device 116(1) may be configured to execute a carbon capture control application 118(1), and the computing device 116(2) may be configured to execute a carbon capture control application 118(2). In general, the carbon capture control application 118, whether executed on computing device 116(1) or on computing device 116(2), may provide a graphical interface to the display of the computing device 116. The graphical interface may include first data and one or more selectable controls accessible by an operator. The first data may include sensor data from the CO₂ capture device 102, and the selectable controls may be accessible by the operator to selectively adjust one or more operating parameters of the CO₂ capture device 102. In response to selection of the one or more selectable options, the computing device 116 may communicate a control signal to the CO₂ capture device 102 to selectively adjust one or more operating parameters of the CO₂ capture device 102. Other implementations are also possible.

The CO₂ capture device 102 may include a control circuit 124, which may be coupled to a plurality of controllable elements 125, which may be associated with the CO₂ capture device 102. The plurality of controllable elements 125 may include blowers, heaters, valves, actuators, chillers, refrigeration components, other components, or any combination thereof. The control circuit 124 may be coupled to a plurality of sensors 132, which may be associated with various elements of the CO₂ capture device 102.

The control circuit 124 may include one or more processors 126, which may process data, and which may execute processor-readable instructions. The control circuit 124 may include or may be coupled to a touchscreen interface 128, a network transceiver 130, and an input/output interface 134, each of which may be coupled to the processor 126. The network transceiver 130 may be configured to couple the control circuit 124 to the network 110. The I/O interface 134 may be coupled to the plurality of controllable elements 125 and to the one or more sensors 132.

In some embodiments, the processor 126 may be configured to receive sensor data related to the processing of the waste stream 102 to produce the purified CO₂ gas 106 from the plurality of sensors 132. The processor 126 may be further configured to generate a graphical interface including data determined from the sensor data and including a plurality of selectable controls and may provide the graphical interface to one or more computing devices 116 or 120 associated with an operator. The operator may view the data and optionally interact with one or more of the plurality of selectable controls to adjust one or more parameters corresponding to one or more of the controllable elements 125. In some implementations, the processor 126 may provide the graphical interface to the touchscreen interface 128 through which an operator may interact with the selectable controls.

In some implementations, the processor 126 may communicate data related to the sensor data to the local server 112, the control and maintenance server 114, the carbon capture control application 118(1) of the computing device 116(1), the carbon capture control application 118(2) of the computing device 116(2), or any combination thereof.

The local server 112 may receive the data from the CO₂ capture device 102 and may store the data. The local server 112 may provide a graphical interface including at least a portion of the data received from the CO₂ capture device 102 and including selectable controls accessible by an operator one of the computing devices 116(1) or 116(2) to adjust operation of the CO₂ capture device 102. In some embodiments, the computing device 116(1) or 116(2) may access the data via the carbon capture control application 118(1) or 118(2), respectively, or may access the data via a webpage presented within an Internet browser application executing on the computing device 116(1) or 116(2). The local server 112 or the control and maintenance server 114 (or optionally the CO₂ capture device 102) may communicate with a customer device, such as computing device 120, to provide environmental or other information related to the operation of the CO₂ capture device 102 to the carbon capture customer application 122. Other embodiments are also possible.

In some embodiments, the local server 112, the control circuit 124, or the control and maintenance server 114 may be configured to automatically adjust one or more parameters of the CO₂ capture device 102 to selectively adjust the purified CO₂ gas production process. Further, manual adjustments may be made by an operator via the computing device 116(1), the computing device 116(2), or the touchscreen interface 128. Other embodiments are also possible.

It should be appreciated that the processor 126 may provide a graphical interface to the touchscreen display 128. Further, it should be appreciated that any of the processor 126, the local server 112, and the control and maintenance server 114 may be configured to provide data, a graphical interface, or both to any of the computing devices 116 and 120. In some examples, such as where the operator is accessing a webpage via an Internet browser application, any of the processor 126, the local server 112, and the control and maintenance server 114 may provide a graphical interface including the data for rendering within the Internet browser application. Other embodiments are also possible.

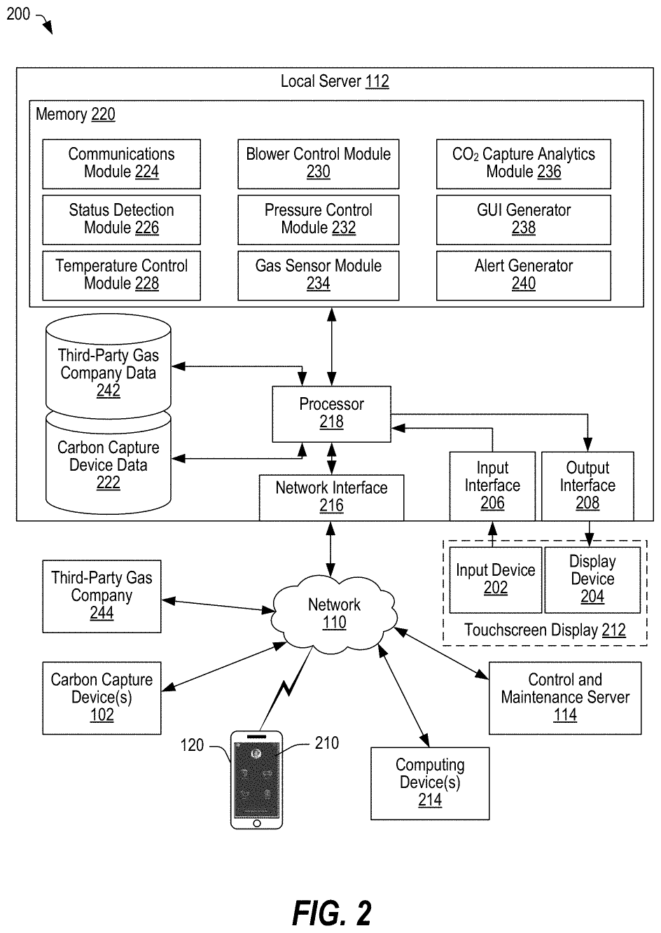

FIG. 2 depicts a block diagram of a system 200 including a local server 112 configured to provide an interface to monitor and control one or more CO₂ capture devices 102, in accordance with certain embodiments of the present disclosure. The system 200 may include all the elements of the system 100 of FIG. 1. The local server 112 may be configured to communicate with a control and maintenance server 114, a computing device 120, a carbon capture device 102, one or more computing devices 214, and other devices through the network 110. The local server 112 may also communicate with a computer server of a third-party gas company 244, web sites, and various corporate systems through the network 110. The computing devices 214 may include smartphones, tablet computers, laptop computers, other computing devices, or any combination thereof. The local server 112 may also communicate with a computing device 120, which may present a graphical interface 210 on its touchscreen display including data about the CO₂ capture process of a CO₂ capture device 102. Other embodiments are also possible.

The local server 112 may be coupled to an input device 202 (such as a keyboard, a touch-sensitive surface, a pointer, a scanner, a microphone, another input device, or any combination thereof) to receive operator input. The local server 112 may be coupled to a display device 204 to display a graphical interface including data and selectable controls. In some implementations, the local server 112 may be coupled to one or more other output devices. In some embodiments, the input device 202 and the display device 204 may be combined to provide a touchscreen display 212. Other embodiments are also possible.

The local server 112 may include a network interface 216 coupled to the network 110. The local server 112 may include a processor 218 coupled to the network interface 216. Further, the local server 112 may include an input interface 206 coupled to the input device 202 and an output interface 208 coupled to the display device 204 (and optionally to other output devices). The processor 218 may be coupled to the input interface 206 and the output interface 208.

The local server 112 may include a memory 220 coupled to the processor 218 and configured to store data and instructions that, when executed, may cause the processor 218 to perform a variety of monitoring, control, and communication functions. The local server 112 may also include databases, such as third-party gas company database 242 and carbon capture device data 222. The third-party gas company database 242 may store data corresponding to companies that might be able to utilize and/or transport purified CO₂ gas generated and stored by the CO₂ capture device 102. The carbon capture device data 222 may include data associated with each of a plurality of CO₂ capture devices 102, including production information, data related to sensor measurements, parameter data, other data, or any combination thereof. The carbon capture device data 222 may include CO₂ data from one or more CO₂ capture devices 102.

The memory 220 may include a communications module 224 that, when executed, may cause the processor 218 to communicate with the CO₂ capture devices 102 and with other devices to receive information, to search for information, and so on. The communications module 224 may cause the processor 218 to communicate with each of a plurality of CO₂ capture devices 102 periodically, according to a pre-determined schedule, or continuously to retrieve the information. In some examples, the communications module 224 may be configured to passively receive the information. In some embodiments, the communications module 224 may cause the processor 218 to receive data, such as sensor measurement data, purified CO₂ gas production data, parameter data, other data from one or more CO₂ capture devices 102, or any combination thereof.

The memory 220 may also include a status detection module 226 that, when executed, may cause the processor 218 to determine a status of various components of at least one of the CO₂ capture devices 102 based on the received data. The status may include a state of a valve (open, closed, stuck, and so on), a state of the purified CO₂ storage device 108, the state of various other components (such as scrubbers, blowers, compressors, valves, actuators, sensors, and so on), or any combination thereof.

The memory 220 may include a temperature control module 228 that, when executed, may cause the processor 218 to determine a temperature of one or more components. Further, in response to determining the temperature, the temperature control module 228 may selectively determine one or more adjustments to parameters associated with components of the associated CO₂ capture device 102 that may be controlled to maintain the temperature within a pre-determined temperature range. In some implementations, the temperature control module 228 may cause the processor 218 to control one or more heating elements associated with desiccant beds or other components to selectively apply heating. Other implementations are also possible.

The memory 220 may also include a blower control module 230 that, when executed, may cause the processor 218 to determine information related to one or more blower components of the CO₂ capture device 102. Additionally, the blower control module 230 may cause the processor 218 to selectively determine one or more adjustments to parameters of the blower components of the associated CO₂ capture device 102 to adjust the output of the blowers.

The memory 220 may also include a pressure control module 232 that, when executed, may cause the processor 218 to determine information related to the fluid pressure at various points within the CO₂ capture process performed by the CO₂ capture device 102. In some embodiments, the pressure control module 232 may be configured to cause the processor 218 to selectively determine one or more adjustments to parameters of one or more components the associated CO₂ capture device 102 to control the fluid pressure.

The memory 220 may also include a gas sensor module 234 that, when executed, may cause the processor 218 to extract the sensor measurement data from the data received from the CO₂ capture device 102. The sensor measurement data may include temperature data, pressure data, moisture data, CO₂ volume data, timing data, other data, or any combination thereof.

Further, the memory 220 may include a CO₂ capture analytics module 236 that, when executed, may cause the processor 218 to process the received data and the data determined by the status detection module 226, the temperature control module 228, the blower control module 230, the pressure control module 232, and the gas sensor module 234 to determine one or more adjustments associated with the CO₂ capture device 102. In some embodiments, the CO₂ capture analytics module 236 may be configured to process data from each of a plurality of CO₂ capture devices 102, to determine parameters and settings that may enhance the CO₂ production for each CO₂ capture device 102 individually and optionally for establishing default settings that may provide an initial approximation for optimal production of purified CO₂ gas from the waste gas stream. In some embodiments, the CO₂ capture analytics module 236 may provide recommended settings and suggested adjustments to an operator via a graphical interface.

The memory 220 may include a graphical user interface (GUI) generator 238 that, when executed, may cause the processor 218 to generate a graphical interface including data related to at least one CO₂ capture device 102 and including selectable controls accessible by an operator to review the data, to adjust one or more operating settings of the CO₂ capture device 102, to communicate with other computing devices, or any combination thereof. The data may be provided in various formats, including text, visualizations, or other data. Further, the selectable controls may include buttons, links, tabs, other selectable elements, or any combination thereof.

In some embodiments, the memory 220 may include an alert generator 240 that, when executed, may cause the processor 218 to monitor one or more parameters of the CO₂ capture device 102 and to selectively generate an alert for transmission to a computing device when, for example, one or more components of the CO₂ capture device 102 are malfunctioning, production falls below a threshold production level, or some other measurement requires an operator's attention. In some implementations, the alert generator 240 may cause the processor 218 to push CO₂ production statistics to an application executing on a computing device, such as the computing device 120 in FIG. 1 (e.g., a smartphone), the computing devices 214, the computing devices 116 in FIG. 1, the control and maintenance server 114, or any combination thereof. Further, the alert generator 240 may cause the processor 218 to send text messages, email messages, voice messages, or any combination thereof (i.e., an alert) in response to determining (from the information determined by the CO₂ capture analytics module 236) and optionally in response to comparing the information to one or more thresholds. In an example, the alert may be sent in response to a parameter exceeding or falling below a threshold. Other embodiments are also possible.

In some implementations, the local server 112 may communicate data related to available CO₂ inventory to one or more computing devices. The CO₂ capture analytics module 236 may cause the processor 218 to determine how much of the available CO₂ inventory may be used internally and how much may be made available for sale. Other implementations are also possible.

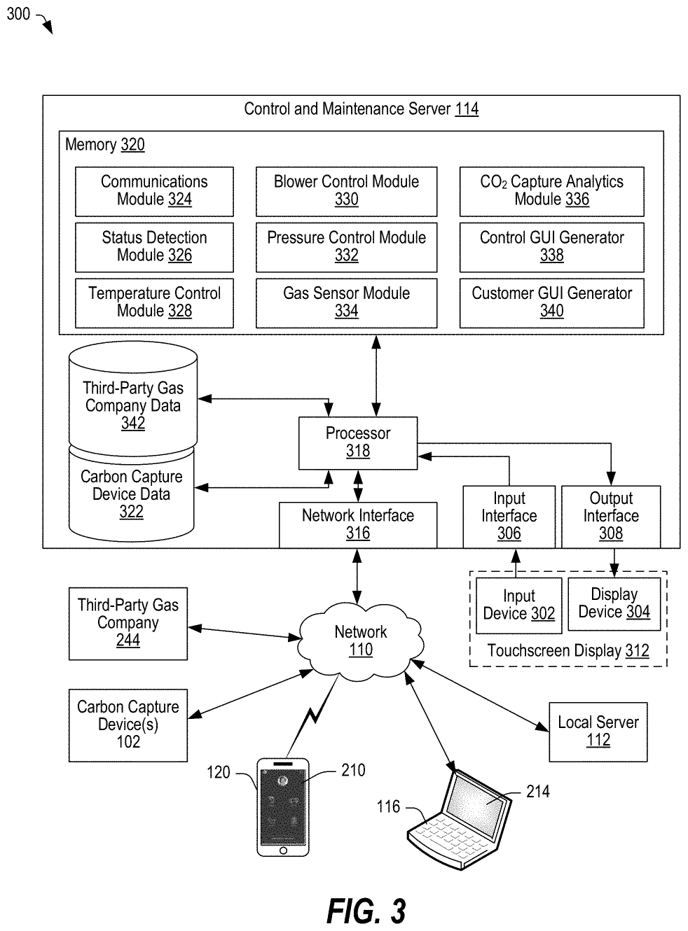

FIG. 3 depicts a block diagram of a system 300 including a control and maintenance server 114 configured to provide an interface to monitor and control a CO₂ capture device 102, in accordance with certain embodiments of the present disclosure. The system 300 may include all the elements of the system 100 of FIG. 1 and the system 200 of FIG. 2. The control and maintenance server 114 may be configured to communicate with one or more CO₂ capture devices 102, one or more computing devices 116 and 120, a local server 112, and one or more third-party gas companies 244.

The control and maintenance server 114 may be configured to interact with the local server 112 and optionally one or more of the CO₂ capture devices 102. In some implementations, the control and maintenance server 114 may determine one or more parameters corresponding to production of the purified CO₂ based on sensor data and other data from a first CO₂ capture device 102 and may communicate one or more parameter adjustments to the first CO₂ capture device 102. The control and maintenance server 114 may also determine one or more parameters corresponding to production of the purified CO₂ based on sensor data and other data from a second CO₂ capture device 102. The control and maintenance server 114 may be configured to adjust one or more operating parameters, to report information, and so on.

In some implementations, the control and maintenance server 114 may also be configured to provide information to computing devices of various individuals, including administrators and customers. The control and maintenance server 114 may include an input interface 306 coupled to one or more input devices 302, such as a keypad, a stylus, a pointer device, a microphone, a scanner, a camera, or another input device. The control and maintenance server 114 may include an output interface 308, which may be coupled to one or more output devices, such a display device 304, a printer, a speaker, another output device, or any combination thereof. In some implementations, the input device 302 and the display device 304 may be combined as a touchscreen display 312.

The control and maintenance server 114 may include a processor 318 coupled to the input interface 306 and to the output interface 308. The control and maintenance server 114 may include a network interface 316 coupled to the processor 318 and configured to couple to the network 110. The control and maintenance server 114 may further include a memory 320 coupled to the processor 318 and may include databases including a third-party gas company database 342 and a carbon capture device database 322, which may be coupled to the processor 318. The third-party gas company database 342 may store data corresponding to companies that might be able to utilize and/or transport purified CO₂ gas generated and stored by the CO₂ capture device 102. The carbon capture device data 322 may store data associated with each of a plurality of CO₂ capture devices 102, including production information, data related to sensor measurements, parameter data, other data, or any combination thereof.

The memory 320 may include a communications module 324 that, when executed, may cause the processor 318 to communicate with the CO₂ capture devices 102. The communications module 324 may cause the processor 318 to communicate with a local server 112 or with other devices to receive information, to search for information, and so on. The communications module 324 may cause the processor 318 to communicate with each of a plurality of CO₂ capture devices 102 periodically, according to a pre-determined schedule, or continuously to retrieve the information. In some examples, the communications module 324 may be configured to passively receive the information. In some implementations, the communications module 324 may cause the processor 318 to receive data, such as sensor measurement data, purified CO₂ gas production data, parameter data, and other data from one or more CO₂ capture devices 102.

The memory 320 may also include a status detection module 326 that, when executed, may cause the processor 318 to determine a status of various components of at least one of the CO₂ capture devices 102 based on the received data. The status may include a state of a valve (open, closed, stuck, and so on), a state of the purified CO₂ storage device 108, the state of various other components (such as scrubbers, blowers, compressors, sensor measurement data, and so on), or any combination thereof.

The memory 320 may include a temperature control module 328 that, when executed, may cause the processor 318 to determine a temperature of one or more components, for example, based on sensor data of the CO₂ capture devices 102. Further, in response to determining the temperature, the temperature control module 328 may selectively determine one or more adjustments to parameters associated with components of the associated CO₂ capture device 102 that may be controlled to maintain the temperature within a pre-determined temperature range. For example, the temperature control module 328 may cause the processor 318 to activate one or more heater elements to supply heat to a desiccant bed, to activate one or more refrigeration elements, and so on.

The memory 320 may also include a blower control module 330 that, when executed, may cause the processor 318 to determine information related to one or more blower components of the CO₂ capture device 102. Additionally, the blower control module 330 may cause the processor 318 to selectively determine one or more adjustments to parameters of the blower components of the associated CO₂ capture device 102 to adjust the output of the blowers.

The memory 320 may also include a pressure control module 332 that, when executed, may cause the processor 318 to determine information related to the fluid pressure at various points within the CO₂ capture process performed by the CO₂ capture device 102. In some embodiments, the pressure control module 332 may be configured to cause the processor 318 to selectively determine one or more adjustments to parameters of one or more components of the CO₂ capture device 102 to control the fluid pressure.

The memory 320 may also include a gas sensor module 334 that, when executed, may cause the processor 318 to extract the sensor measurement data from the data received from the CO₂ capture device 102. For example, the gas sensor module 334 may cause the processor 318 to determine a concentration of CO₂ gas at an input of the CO₂ capture device 102 and may selectively activate the CO₂ capture device 102 to process the waste gas stream when the concentration of CO₂ is above a threshold CO₂ concentration. Other implementations are also possible.

Further, the memory 320 may include a CO₂ capture analytics module 336 that, when executed, may cause the processor 318 to process the received data and the data determined by the status detection module 326, the temperature control module 328, the blower control module 330, the pressure control module 332, and the gas sensor module 334 to determine one or more adjustments associated with the CO₂ capture device 102. In some embodiments, the CO₂ capture analytics module 336 may be configured to process data from each of a plurality of CO₂ capture devices 102, to determine parameters and settings that may enhance the CO₂ production for each CO₂ capture device 102 individually and optionally for establishing default settings that may provide an initial approximation for optimal production of purified CO₂ gas from the waste gas stream. In some embodiments, the CO₂ capture analytics module 336 may provide recommended settings and suggested adjustments to an operator via a graphical interface.

The memory 320 may further include a control GUI generator 338 that, when executed, may cause the processor 318 to generate a graphical interface that may be provided to any of the computing devices 116 (in FIG. 1), 120, and 214, to another device, or any combination thereof to facilitate control of the CO₂ capture device 102, remotely. The graphical interface may include data corresponding to parameters of a CO₂ capture device 102 and may include selectable options accessible by an operator to selectively adjust one or more of the parameters. Other embodiments are also possible.

The memory 320 may also include a customer GUI generator 340 that, when executed, may cause the processor 318 to generate a graphical interface including data related to the operation of the CO₂ capture device 102 and to provide the graphical interface to a computing device 116 or 120, such as a smartphone, associated with a customer. The control and maintenance server 114 may determine proximity of the customer's computing device 116 and may communicate the graphical interface to the computing device 116 when the computing device 116 is within a pre-determined distance from a facility. Other implementations are also possible.

In some embodiments, the system 100 in FIG. 1, the system 200 in FIG. 2, and the system 300 in FIG. 3 may be implementations of the same system. In an example, the systems 100, 200, and/or 300 may provide a real-time network of hardware, software, and sensors configured to enable remote monitoring, analyzing, measuring, communicating, impacting, and controlling a CO₂ gas capture process. The systems 100, 200, and 300 may include a piece of field equipment (e.g., the CO₂ capture device 102) configured to CO₂ gas from a waste stream of mixed gas.

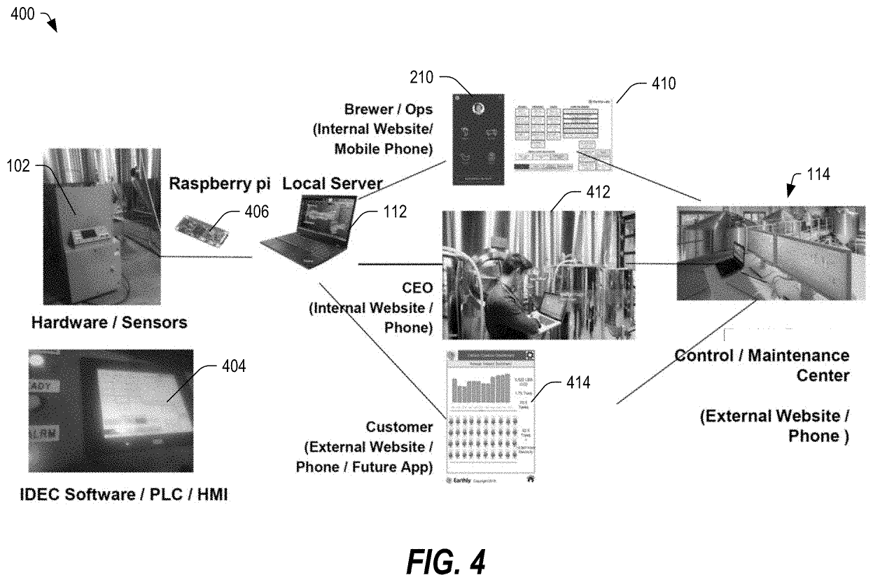

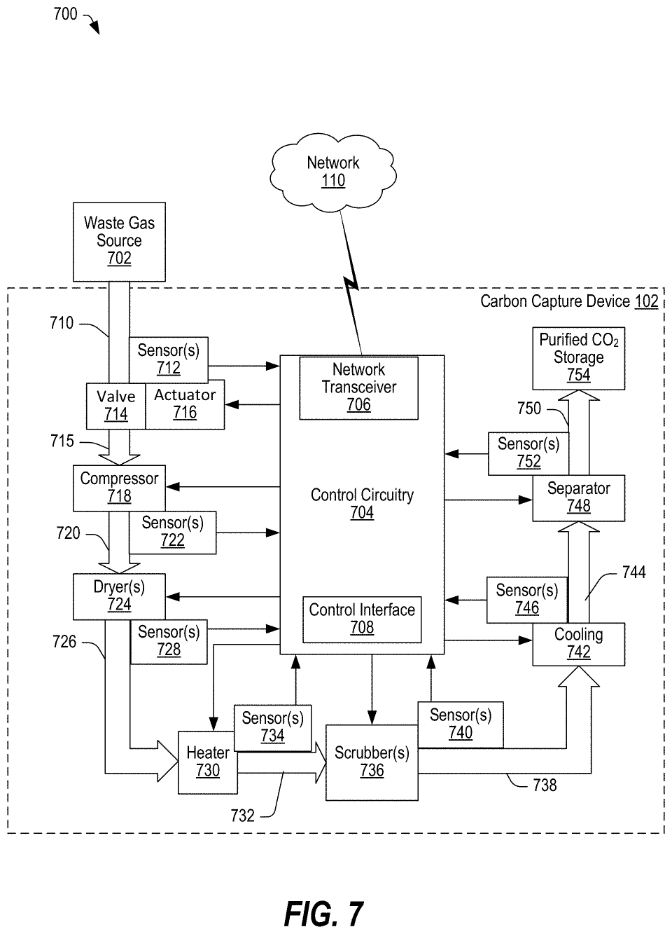

The CO₂ capture device 102 may include sensors as described below with respect to FIGS. 4 and 7 to capture data corresponding to the CO₂ capture process. The data may include measurement data corresponding to a plurality of chemical process input parameters and a set of chemical process output parameters. The circuit of the CO₂ capture device 102 (or of the local server 112 or the control and maintenance serer 114) may be configured to determine a set of desired chemical process output parameters. The CO₂ capture device 102 may determine adjustments to one or more of the input process parameters and the output process parameters to achieve the desired chemical process output parameters. Further, the CO₂ capture device 102 may communicate measurement data to one or more remote devices to enable remote monitoring and analysis of the CO₂ capture process. In some embodiments, the application executing on the computing devices may include a version having an open Application Programming Interface (API) that may allow customers to import data to a website and to process the data to track impact in real-time.

In some implementations, the systems 100, 200, and/or 300 may allow any piece of remote field equipment that performs complex chemical processing to be monitored, controlled, and operated remotely. Further, the control and maintenance server 114 or the local server 112 may enable an array of distributed field equipment (e.g., CO₂ capture devices) situated in different places (such as around the world) to be controlled primarily through a graphical interface executing on a computing device.

In some implementations, a system may include remotely monitoring, controlling, analyzing, and sharing a separation process of a (fermentation) or CO₂ gas stream that removes water, oxygen, and volatile organic compounds (VOCs) from a waste stream to produce a controlled product stream. The system may include a plurality of field equipment for performing the CO₂ capture process. The field equipment may be adapted to be responsive to electrical signals resulting in control of a plurality of process parameters. The system may include a server including a hardware processor and a memory that may store data and processor-executable instructions. The system may further include a communications-link between the server and one or more pieces of field equipment.

In some embodiments, the instructions stored in the memory, when executed, may cause the processor to establish a communications link between the field equipment and the server and to establish a client-server communications link between a user device and the server. Further, the instructions may cause the processor to provide a graphical interface to a display of a computing device through a network. The graphical interface may present data related to a plurality of process parameters within the graphical interface on the display of the computing device. The instructions may also cause the processor to receive a set of separation process input parameters corresponding to parameters of an input stream of the CO₂ gas; receive a set of desired process output parameters corresponding to desired parameters of an output chemical stream comprising a CO₂ stream to control oxygen in the product streams; and selectively controlling a set of separation process control parameters to achieve the desired chemical process output parameters either automatically based on the input parameters and desired output parameters or in response to operator inputs.

In some implementations, the CO₂ capture device 102 may control the CO₂ capture process by monitoring oxygen, CO₂, and other components within the waste stream to determine suitability to initiate (or continue) the CO₂ capture operation. In response to relatively high oxygen content, the CO₂ capture device may determine a relatively low CO₂ content in the gas stream. Further, the CO₂ capture device 102 may determine and optionally control an inlet flow rate for the separation process, where the inlet flow rate may be controlled by an inlet control valve of the CO₂ capture device 102. Further, the CO₂ capture device 102 may be configured to determine a system operating pressure for the separation process, which system operating pressure may be controlled by a pressure control valve. Additionally, the CO₂ capture device 102 may be configured to determine a temperature set-point for the separation process, which temperature set-point may be controlled by a temperature controller. In some implementations, the CO₂ capture device 102 may adjust the inlet flow rate by controlling the inlet control valve, the system operating pressure by controlling the pressure control valve, and the temperature set-point by adjusting one or more of heating elements or cascade refrigeration elements to maintain a desired temperature range. In some implementations, the CO₂ capture device 102 may provide a graphical interface (i.e., a human-machine interface (HMI)) to allow supervisory intervention and specification of operating points to allow an operator to manually control the set of separation process control parameters.

In some implementations, the separation process may include separating CO₂ from a mixed waste gas stream including CO₂ gas stream to improve purity by removing water, oxygen, and volatile organic compound (VOC) products from the output product stream. The resulting CO₂ output stream 106 may be a food-grade, purified CO₂ output that may be stored in a liquid phase.

In some implementations, the memory of the CO₂ capture device 102 may include instructions that, when executed, may cause the processor to control an inlet flow rate of the raw CO₂ gas stream to a track a predetermined inlet pressure and to control a temperature set-point of a separation subsystem. The inlet flow rate, the system operating pressure, and the temperature set-point may be controlled to maintain a pure CO₂ stream.

In some implementations, the communications link between the CO₂ capture device 102 and the local server 112 (or the control and maintenance server 114) may utilize a communication protocol selected from the group consisting of Modbus, CAN bus, TCP/IP, UDP, 3G, 4G, LTE, coaxial, IEEE 802.11a/b/g/n/x, IEEE 802.15.4, Bluetooth®, virtual private network (VPN), Internet Protocol security (IPsec), Internet Security Association and Key Management Protocol (ISAKMP), near-field communication, Fieldbus, 900 MHz radio, or any combination thereof. Additionally, in some implementations, the instructions may cause the processor to perform a process of controlling separation of a raw CO₂ gas stream by controlling one or more process parameters. The one or more process parameters may include an inlet flow rate of a raw natural gas stream, a system operating pressure, and a temperature set-point of a separation subsystem. The one or more process parameters may be controlled to maintain the desired CO₂ product temperature. In some embodiments, the system operating pressure and the temperature set-point may be determined by one or more input parameters selected from the group including a desired minimum oxygen content of the raw natural gas stream and a volume flow rate of the raw CO₂ gas stream. In some embodiments, the CO₂ capture device 102 may control the inlet flow rate by controlling an inlet control valve or a compressor speed of one or more compressors.

In some implementations, the CO₂ capture device 102 may apply a timestamp to each measurement. In an example, the CO₂ volume measurements may be provided in various time measurements to allow customers (or customer devices) to analyze capture rates by time-interval, such as by minute, day, week, month, year, cumulative month-to-date, cumulative year-to-date, another time increment, or any combination thereof. Furthermore, this data may be spliced in various visual formats, including real-time data, monthly analysis, annual impact reports, other reports, or any combination thereof. In some embodiments, the CO₂ volume data may be further analyzed for environmental impact, and the data may be compared against CO₂ emissions reduction targets at organization, city, State, Federal or international levels. The volume may be converted to compare environmental impacts (tree equivalents), economic savings, or other impacts.

In some implementations, the data may be presented within a graphical interface, which may be viewed on a mobile phone (i.e., a smartphone), a tablet computer, a laptop computer, video screen, a touchscreen, another electronic device, or any combination there. In some implementations, the data APIs may enable data sharing with multiple users, from system owners to consumers. Further, the data related to CO₂ emissions captured and avoided may be used to facilitate reporting to local, state, or federal agencies to secure CO₂ emission reduction tax credits or rebates. Other implementations are also possible.

FIG. 4 depicts a diagram of components of a system 400 including multiple interfaces for monitoring and controlling a CO₂ capture device 102, in accordance with certain embodiments of the present disclosure. The CO₂ capture device 102 may include circuitry including a plurality of sensors and including a control circuit 406. In one possible embodiment, the control circuit 406 may include a Raspberry PI®, which is a commercially available circuit product produced by the Raspberry Pi Foundation of the United Kingdom.

The CO₂ capture device 102 may include a display interface, such as a touchscreen display 404, which may be configured to display a graphical interface including data and selectable options accessible by an operator to selectively adjust one or more parameters. The CO₂ capture device 102 may communicate data, such as sensor data and other data corresponding to the production of purified CO₂ gas, to a local server 112.

In this example, the local server 112 may communicate a graphical interface 210 including data 410 and selectable options accessible by a brewer (or other operator) to review and optionally operation of the CO₂ capture device 102. The local server 112 may also communicate a graphical interface including data to employees of a company, such as a corporate officer, as shown at 412. The local server 112 may also provide data to a graphical interface of a customer device (e.g., a smartphone, another computing device, or any combination thereof) or to an external website.

The system 400 also includes a control and maintenance system 114, which may be configured to provide the data to the graphical interface 210, to the company employee at 412, or to the customer device 414. The control and maintenance system 114 may be coupled to the CO₂ capture device 102 to receive CO₂ capture data and to provide a graphical interface including data related to the captured CO₂ to customer device 414 or to another computing device. Other embodiments are also possible.

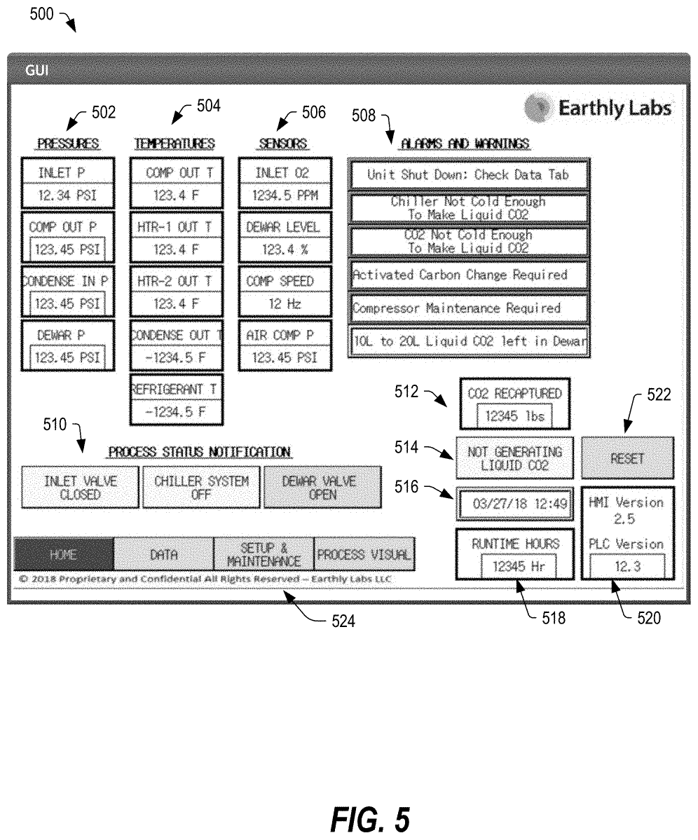

FIG. 5 depicts a graphical interface of a CO₂ capture device 500 including collected data associated with a CO₂ capture device 102, in accordance with certain embodiments of the present disclosure. The graphical interface 500 may include data corresponding to measurements by a plurality of pressure sensors, including a plurality of pressure measurements 502, including an inlet pressure (INLET P), an outlet pressure (COMP OUT P), a condenser pressure (CONDENSE IN P), a Dewar pressure (DEWAR P), other pressures, or any combination thereof. The graphical interface 500 may further include data corresponding to a plurality of temperature measurements 504, including a compressor outlet temperature (COMP OUT), heater output temperatures (HTR-1 OUT T, HTR-2 OUT T, etc.), condenser output temperatures (CONDENSE OUT T), refrigerant temperatures (REFRIGERANT T), other temperatures, or any combination thereof.

The graphical interface 500 may also include other sensor data corresponding to other sensors 506, such as an inlet O₂ content data (INLET O2), a Dewar level data (DEWAR LEVEL), a compressor speed data (COMP SPEED), an air compressor pressure data (AIR COMP P), other sensor data, or any combination thereof. Further, the graphical interface 500 may include data including a plurality of alarms and warnings, which may be generated automatically in response to comparing measurement data to one or more thresholds. The alarms and warnings may include a “Unit Shut Down: Check Data Tab” warning, a “Chiller Not Cold Enough to Make Liquid CO2” warning, a “CO2 Not Cold Enough” warning, an “Activated Carbon Change Required” warning indicating a change to the scrubber/filters is needed, a “Compressor Maintenance Required” warning, and a “10 L to 20 L Liquid CO2 left in Dewar” warning. Other alarms and warnings may also be provided.

The graphical interface 500 may also include a plurality of process status notifications 510, including an “Inlet Valve Closed” notification, a “Chiller System Off” notification, and a “Dewar Valve Open” notification. Though only three notifications are show, other notifications may also be included.

The graphical interface 500 may also include data including an indicator identifying an amount of CO2 that has been recaptured, at 512 (CO2 RECAPTURED). The graphical interface 500 may also include a “Not Generating Liquid CO2” indicator 514, a date and time stamp 516, and a RUNTIME HOURS indicator 518 corresponding to a number of runtime hours of the CO₂ capture device 102. The graphical interface 500 may further include a “Reset” button 522 and version information 520 corresponding to the software executing on the circuitry of the CO₂ capture device 102 (e.g., HMI Version 2.5 and PLC Version 12.3).

The graphical interface 500 may also include a plurality of selectable controls 524 accessible by the operator to access one or more screens of the graphical interface 500. In this example, the one or more selectable controls 524 may be buttons, tabs, or other types of controls. In the illustrated example, the “Home” selectable control 524 is selected, and the data, alarms, and notifications displayed are associated with the “Home” screen.

Other selectable controls 524 may include a “Data” control option accessible by the operator to view measurement and production data and optionally to review the data in one or more selectable visualizations, including bar graphs, pie charts, line graphs, tables, other visualizations, or any combination thereof. The graphical interface 500 may include a “Setup and Maintenance” control option accessible by an operator to adjust one or more settings, such as alerting thresholds and the like, and optionally to access options corresponding to maintenance operations. The graphical interface 500 may also include a “Process Visual” control option accessible by an operator to view a visual representation of the components of the CO₂ capture device 102 or other visualizations.

It should be appreciated that the graphical interface 500 represents one possible example of a graphical interface including data and selectable options. Other arrangements of the data and other selectable options are also possible.

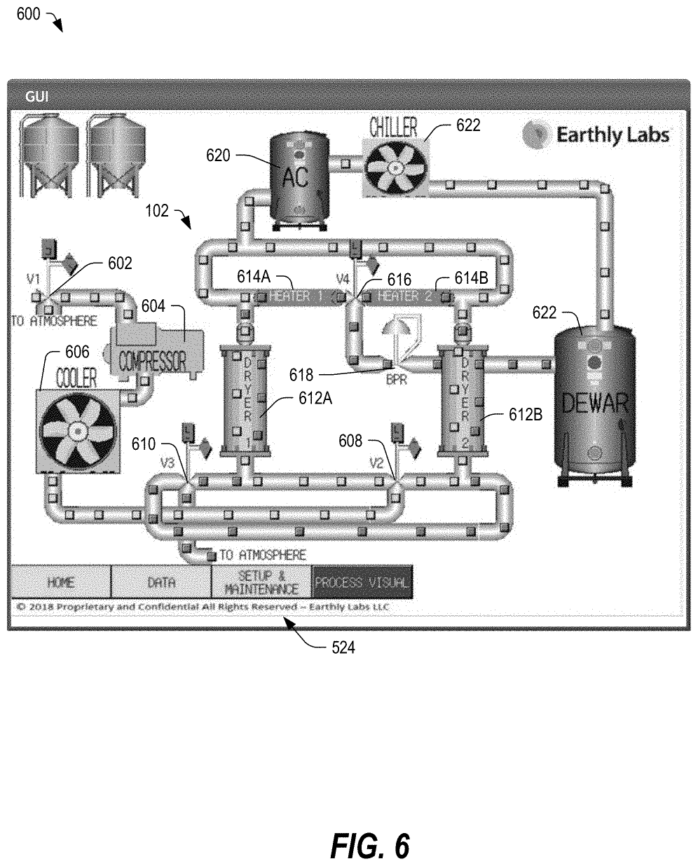

FIG. 6 depicts a graphical interface 600 of a CO₂ capture device 102 including a graphical representation of the CO₂ capture device 102, in accordance with certain embodiments of the present disclosure. The graphical interface 600 may include selectable controls 524, and in this example the “Process Visual” control option is selected, causing the graphical interface 600 to display a representation of components of the CO₂ capture device 102.

Within the graphical interface 600, the CO₂ capture device 102 may include an inlet valve 602 configured to couple a waste gas stream to an input port of a compressor 604, which may include an outlet port coupled to an input port of a chiller 606. The chiller 606 may include an output port coupled to a first dryer 612(1) and a second dryer 612(2) through valves 608 and 610, respectively. The outputs of the dryers 612(1) and 612(2) may be coupled to an air conditioner 620 through a valve 616 and through heaters 614(1) and 614(2). The CO₂ capture device 102 may further include a backflow valve 618. The air conditioner 620 may include an output coupled to an input of a chiller 622, which may chill the CO₂ gas into a liquid phase for storage within the Dewar 624.

It should be appreciated that the CO₂ capture device 102 includes a plurality of sensors and a plurality of controllable components, each of which may be coupled to control circuitry. The sensors may be distributed across multiple components of the CO₂ capture device 102 to sense a variety of parameters, including oxygen content, moisture levels, temperature, pressure, and so on. In some implementations, the sensors may be represented on the visualization within the graphical interface 600 and may be presented with different colors, depending on the state of the sensed parameters. Other implementations are also possible. An example of the CO₂ capture device 102 including sensors, controllable components, and a control circuit is described below with respect to FIG. 7.

FIG. 7 depicts a block diagram 700 of a CO₂ capture device 102 including a plurality of sensors and controllable components, in accordance with certain embodiments of the present disclosure. The CO₂ capture device 102 may be coupled to a waste gas source 702 to receive a waste gas stream of mixed gases including CO₂. In some implementations, the waste gas source 702 may be a vent of a fermentation tank, a pipeline from an industrial process, another source, or any combination thereof.

The CO₂ capture device 102 may include control circuitry 704 including a network transceiver 706 configured to communicate with the network 110. The CO₂ capture device 102 may further include a processor configured to receive sensor data and to selectively generate control signals. Further, in some embodiments, the CO₂ capture device 102 may include a control interface 708, such as a touchscreen interface configured to display data and selectable options and accessible by an operator to control one or more components and to control operation of the CO₂ capture device 102.

The CO₂ capture device 102 may include an inlet 710 coupled to the waste gas source 702 to receive the mixed gas waste stream. The CO₂ capture device 102 may further include one or more sensors 712 coupled to the inlet 710 and to the control circuitry 704. The one or more sensors 712 may include oxygen sensors, moisture sensors, contaminant sensors, temperature sensors, other sensors, or any combination thereof. The one or more sensors 712 may detect parameters of the waste stream within the conduit 710. The one or more sensors 712 may be configured to communicate one or more sensor signals proportional to one or more parameters to be measured. In an example, at least one of the one or more sensors 712 may be configured to measure the content of CO₂ within the waste gas stream received at the inlet 710. The CO₂ capture device 102 may include a valve 714 coupled between the inlet 710 and a compressor 718. The valve 714 may be controlled by an actuator 716 to open and close the valve 714 in response to control signals from the control circuitry 704. The actuator 716 may include a control input coupled to the control circuitry 704 to receive a control signal. The output of the valve 714 may be coupled to a conduit 715.

The CO₂ capture device 102 may further include a compressor 718 including a first input coupled to the conduit 715 to receive at least a portion of a waste gas stream and including a second input coupled to the control circuitry 704 to receive an electrical signal. The compressor 718 may be configured to compress the waste gas stream to a selected pressure based on control signals from the control circuitry 704. The compressor 718 may further include an output coupled to a conduit 720.

The CO₂ capture device 102 may also include one or more sensors 722. The one or more sensors 722 may be coupled to the compressor 718 (including the input of the compressor 718, the output of the compressor 718, and the compressor itself). The one or more sensors 722 may also be coupled to the conduit 726. The one or more sensors 722 may be coupled to the control circuitry 704 and may be configured to send signals to the control circuitry 704 that are proportional to a parameter to be measured. In some implementations, the one or more sensors 722 may measure parameters associated with the compressed waste stream within the conduit 720, within the compressor 718, or within the conduit 715. Other implementations are also possible.

The CO₂ capture device 102 may include one or more dryers 724 coupled to the conduit 720 and coupled to the control circuitry 704. The one or more dryers 724 may include one or more desiccant beds, one or more blowers, one or more heating elements, one or more other components, or any combination thereof.

The CO₂ capture device 102 may also include a conduit 726 coupled to an output of the one or more dryers 724. The CO₂ capture device 102 may include one or more sensors 728, which may be coupled to the one or more dryers 724, the conduit 720, the conduit 726, or any combination thereof. The one or more sensors 728 may measure one or more parameters of the waste stream. The one or more sensors 728 may be coupled to the control circuitry 704 to provide signals proportional to a parameter to be measured.

The CO₂ capture device 102 may also include a heater 730 including a first input coupled to the conduit 726 to receive the compressed and dried waste stream. In some implementations, the heater 730 may deliver heat to a portion of the waste stream. The heater 730 may also include a second input coupled to the control circuitry 704. The CO₂ capture device 102 may further include a conduit 732 coupled to an output of the heater 730. The CO₂ capture device 102 may further include one or more sensors 734 coupled to at least one of the heater 730, the conduit 732, or any combination thereof. The one or more sensors 734 may include an input coupled to the control circuitry 704. The one or more sensors 734 may be configured to provide signals indicative of a parameter corresponding to the waste stream.

The CO₂ capture device 102 may include one or more scrubbers 736 including an input coupled to the conduit 732. The one or more scrubbers 736 may include a second input coupled to the control circuitry 704 and responsive to signals from the control circuitry 704 to control one or more elements associated with scrubbers 736. In some implementations, the scrubbers 736 may include one or more desiccant beds, filters, or other components. The one or more scrubbers 736 may include one or more outputs coupled to a conduit 738. The CO₂ capture device 102 may include one or more sensors 740 that may be coupled to the conduit 732, the one or more scrubbers 736, the conduit 738, or any combination thereof.

The CO₂ capture device 102 may also include a cooling component 742 including an input coupled to the conduit 738 and including an output coupled to a conduit 744. The cooling component 742 may include a control input coupled to and responsive to signals from the control circuitry 704 to control a refrigeration operation. The cooling component 742 may include an auto-cascade refrigeration unit, which may be a single-condenser system containing a mixture of refrigerants that may approximate the behavior of a conventional cascade refrigeration system in a small form-factor, and which may achieve liquefaction of recovered CO₂.

The auto-cascade refrigeration system may include a closed-loop refrigeration cycle that relies on a single refrigeration compressor as its prime mover. The working fluid in an auto-cascade refrigeration system may include a mixture of refrigerants, which may be either flammable or nonflammable, as required by the process requirements or safety standards associated with particular installations. In some implementations, the auto-cascade refrigeration system may include a mixture of nonflammable refrigerants in an auto-cascade refrigeration cycle. The condenser used in the auto-cascade refrigeration cycle may be air-cooled, which may remove the need for a chilled glycol utility stream or other refrigerant stream. By using air-cooling, the burden on already-overloaded glycol or other cooling systems in craft breweries may be reduced. Moreover, the air-cooling of the auto-cascade refrigeration system may facilitate a mobile, modular system that may be used in small facilities (such as microbreweries or small business) while providing a few input/output connections and few process utility requirements. In some implementations, the mixture of refrigerants may result in development of a two-phase vapor-liquid system inside the refrigeration system. In some implementations, the liquid-phase of the captured CO₂ may be provided to a pressure reducing device within the cooling element(s) (such as the auto-cascade refrigeration system). The pressure reduction may provide a further cooling effect in which the CO₂ stream exchanges heat with the vapor stream to partially condense the refrigerant in preparation for the final refrigeration evaporator heat exchanger. The auto-cascade configuration provides a lower pressure operating point as compared to conventional CO₂ recovery technology. The auto-cascade refrigeration system may achieve low process temperatures, ranging anywhere from −80 to −30° Celsius, colder, or warmer depending on the implementation.

In some implementations, the cooling elements 742 may be formed of separate but connected refrigeration stages, each of which have a primary refrigerant. The refrigerants of each of the stages may work in concert to cause the CO₂ to reach selected temperature. In some implementations, the cooling elements 742 may comprise two separate circuits, each using refrigerants appropriate for its temperature range. The two circuits may be thermally coupled by a cascade condenser, which may be the condenser of the low-temperature circuit and the evaporator of the high-temperature circuit. In an implementation, the refrigerant for the high-temperature circuit may be a low-pressure refrigerant that may include R-22, ammonia, R-507, R-404a, other refrigerants, and so forth. For the low-temperature circuit, the refrigerant may include a high-pressure refrigerant with a high vapor density (even at low temperatures), such as ethylene.

In an example, the condenser of the first stage, called the “first” or “high” stage, may be fan-cooled by the ambient air, or in some implementations, a liquid coolant may be used. The evaporator of the first stage may be used to cool the condenser of the second stage, called the “second” or “low” stage. The unit that makes up the evaporator of the first stage and the condenser of the second stage may be referred to as an “inter-stage” or “cascade condenser.” The two stages may be connected in series to achieve temperatures as low as −85° Celsius. In some implementations, the first stage may be the warmer stage of the cascade refrigeration system and may not exchange heat with the process gas in a pre-cooling step. Instead, the first stage may—exchange heat with the lower temperature cascade refrigeration loop and may lose heat through an air-cooled condenser. The colder stage is the refrigeration loop which may exchange heat with the process gas, achieving a process temperature suitable for liquefaction of CO₂ in a single compact heat exchanger.

In some implementations, the auto-cascade refrigeration system of the cooling elements 742 may utilize a single compressor system that may achieve temperatures as low as −100° Celsius. The auto-cascade refrigeration system may be a complete, self-contained refrigeration system in which multiple stages of cascade cooling effect may occur substantially simultaneously by means of vapor-liquid separation and adiabatic expansion of various refrigerants. Physical and thermodynamic features, along with a series of counterflow heat exchangers and an appropriate mixture of refrigerants, may cooperate to enable the system to reach low temperatures. The auto-cascade refrigeration system may include a vapor compressor, an external air- or water-cooled condenser, a mixture of refrigerants with descending boiling points, and a series of insulated heat exchangers.

As a result of its multiple refrigerants, in some implementations, the cooling elements 742 may achieve a selected temperature in a single stage auto-cascade refrigerator may replace a two-stage (or more) cascade refrigerator. The single-stage auto-cascade refrigerator may be simpler and cheaper to build and operate than conventional refrigeration systems.

In some implementations, the temperatures reached by the auto-cascade refrigeration system may be altered by changing a composition of the mix of refrigerants. Depending a variety of conditions that vary between installations, including higher air contamination, elevated operating pressures, and excessive electricity rates, the cooling elements 742 of the CO₂ capture device 102 may be tuned to reach appropriate temperatures for effective operation in an environment where the CO₂ capture device 102 is or will be installed.

The CO₂ capture device 102 may further include one or more sensors 746, which may be coupled to the conduit 738, the cooling elements 742, and the conduit 744. The one or more sensors 746 may include temperature sensors, pressure sensors, contaminant sensors, CO₂ sensors, 02 sensors, other sensors, or any combination thereof. The one or more sensors 746 may determine parameters of the CO₂ capture process, including one or more temperatures associated with the cooling elements 742, CO₂ sensors, other sensors, or any combination thereof.

The CO₂ capture device 102 may include a separator 748 including an input coupled to the conduit 744 and an output coupled to a purified CO₂ storage 754 via a conduit 750. The CO₂ capture device 102 may include one or more sensors 752 coupled to the conduit 744, the conduit 750, the separator 748, or any combination thereof. The one or more sensors 752 may measure one or more parameters of the process stream. The sensors 752 may be coupled to the control circuitry 704.

The CO₂ capture device 102 may also include a conduit 750 coupled to an output of the separator 748 and coupled to an inlet of the purified CO₂ storage 754. The CO₂ capture device 102 may further include one or more sensors 752 coupled to at least one of the separator 748, the conduit 750, and the purified CO₂ storage 754. Further, the one or more sensors 752 may be coupled to the control circuitry 704.

It should be appreciated that the CO₂ capture device 102 may include a plurality of valves and a plurality of actuators, such as valve 714 and actuator 716 (which are omitted from FIG. 7 for simplicity). Additionally, the CO₂ capture device 102 is shown to include a plurality of sensors 712, 722, 728, 734, 740, 746, and 752. Such sensors may include temperature sensors, pressure sensors, moisture sensors, O₂ sensors, CO₂ sensors, speed sensors, level or volume sensors, activated carbon status sensors, other sensors, or any combination thereof.

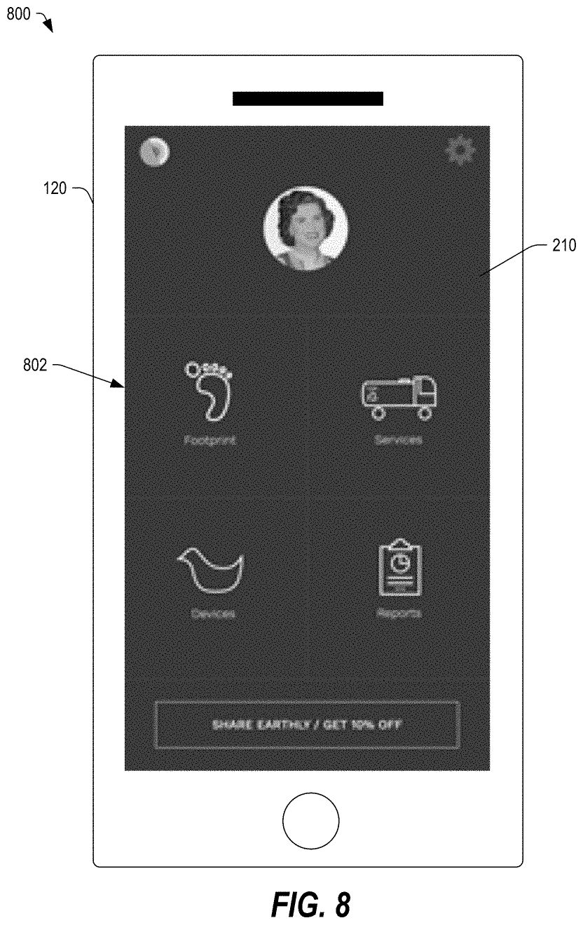

FIG. 8 depicts a diagram 800 including a device 120 having a graphical interface 210 accessible by an operator to view data associated with a CO₂ capture device 102, in accordance with certain embodiments of the present disclosure. The graphical interface 210 may include selectable controls 802 accessible by a user to view “Carbon Footprint” data, “Services”, “Devices”, and “Reports.” Further, the graphical interface 210 may include a “Share” button accessible by a user to share the interface and associated data.

In some implementations, the graphical interface 210 may include selectable controls 210 that may be accessed by a user to view data and selected visualizations. For example, the user may access a “Footprint” control accessible by the user to view a CO₂ footprint for a business that is using the CO₂ capture device 102. The CO₂ footprint may include one or more visualization controls that may be accessed by the user to change from a first visualization of the footprint data to a second visualization.

The graphical interface 210 may include a “Services” control accessible by the user to view one or more services that may be accessed or viewed by the user. The graphical interface 210 may also include a “Devices” control accessible by the user to view one or more devices coupled to the system. The graphical interface may include a “Reports” control accessible by the user to view one or more reports about the CO₂ production. Other implementations are also possible.

In some implementations, the graphical interface 210 may include a “Share Earthly/Get 10% Off” control accessible by the user to recommend the application to one or more other users. The control may also provide an incentive for the customer to share with other customers and friends. Other implementations are also possible.

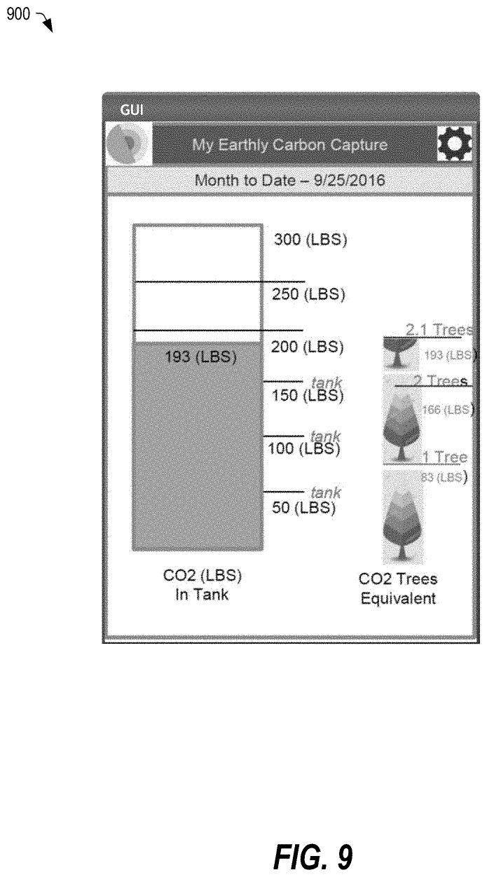

FIG. 9 depicts a graphical interface 900 accessible by an operator to view data associated with a CO₂ capture device 102, in accordance with certain embodiments of the present disclosure. The graphical interface 900 may include a plurality of visualizations of data determined by the CO₂ capture device 102 representing the captured CO₂ gas for a given month (“to date”). In this example, the graphical interface 900 includes a bar graph of the contents of the CO₂ tank. Further, the graphical interface 900 includes a graphical depiction of equivalent of the captured CO₂ gas in terms of the equivalent number of trees. Other embodiments of a graphical interface and other visualizations of the data are also possible.

In this example, the captured CO₂ is represented in terms of pounds (LBS) of captured CO₂. Here, a tree is represented by an equivalent weight of CO₂. Eighty-three pounds of CO₂ are represented as equivalent to the amount of CO₂ that may be absorbed by a tree. Other implementations and other visualizations are also possible.

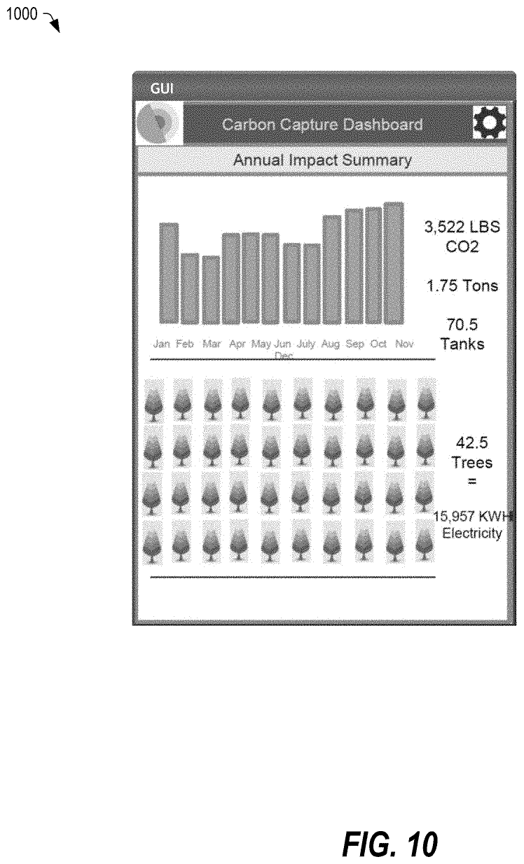

FIG. 10 depicts a graphical interface 1000 accessible by an operator to view data associated with a CO₂ capture device 102, in accordance with certain embodiments of the present disclosure. The graphical interface 1000 may include a bar graph showing the volume of captured CO₂ gas broken down by month. Further, the graphical interface 1000 may include a graphical representation of the equivalent number of trees broken down by month or captured over the course of the year. Other implementations of a graphical interface and other visualizations of the data are also possible.

In this example, the amount of captured CO₂ is also quantified in terms of pounds, tons, number of tanks, and so on. Further, the visualization indicates that the amount of captured CO₂ is equivalent to 42.5 trees. Other implementations are also possible.

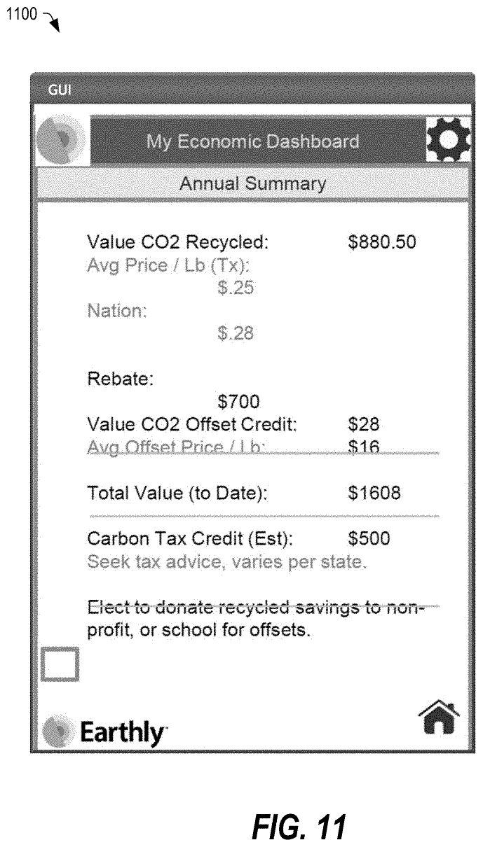

FIG. 11 depicts a graphical interface 1100 accessible by an operator to view data associated with a CO₂ capture device 102, in accordance with certain embodiments of the present disclosure. The graphical interface 1100 may include an economic dashboard depicting an annual summary of the value of the recycled CO₂ gas. Other embodiments of a graphical interface and other visualizations of the data are also possible.

In this example, the value of the recycled CO₂ may be quantified based on the average price per pound of captured CO₂ in Texas ($0.25) and nationally ($0.28). The graphical interface 1100 may also depict the value of available rebates, including the value of CO₂ offset credits, the average offset price per pound, and the total value to date. Further, the graphical interface 1100 may include a carbon tax credit estimate value. In this instance, the estimated carbon tax credit may be $500. Other implementations are also possible.

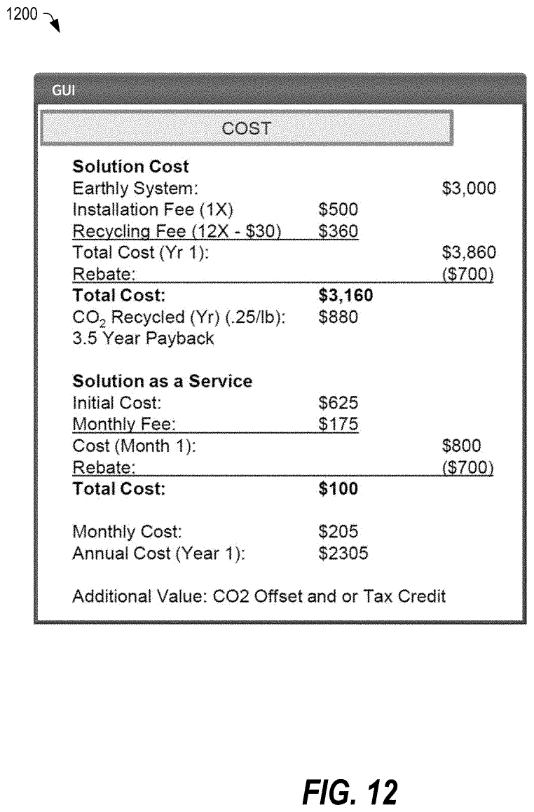

FIG. 12 depicts a graphical interface 1200 accessible by an operator to view data associated with a CO₂ capture device 102, in accordance with certain embodiments of the present disclosure. The graphical interface 1200 may include a cost dashboard depicting the cost of the CO₂ device installation and the cost of operating the device and the CO₂ gas recovery as a service. Other embodiments of a graphical interface and other visualizations of the data are also possible.

In this example, the graphical interface 1200 may depict a solution cost for installation of an Earthly Labs system ($3,000) plus an installation fee ($500) and a recycling fee ($360), which sum to $3,860. The graphical interface depicts an estimated rebate of $700, resulting in a total installation cost of $3,160.

The graphical interface 1200 may also include information regarding the value of the recycled CO₂ per year at an estimated $0.25 per pound (LB). The graphical interface 1200 may also include the initial costs and monthly fees for providing the solution as a service, showing a one-month cost of about $800. The graphical interface 1200 may further include associated rebate data reflecting total costs, as well as the additional values for CO₂ offsets, tax credits, or any combination thereof.

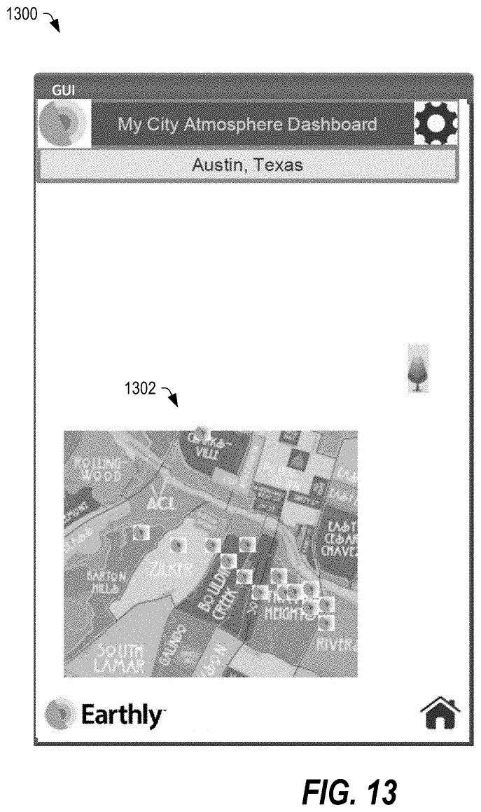

FIG. 13 depicts a graphical interface 1300 accessible by an operator to view map data 1302 associated with one or more CO₂ capture devices 102, in accordance with certain embodiments of the present disclosure. The map data 1302 may include a plurality of markers or indicators 1304 representing the geographic locations where the CO₂ capture devices 102 are located within a geographic area. The plurality of markers or indicators 1304 may change color in response to selection by an operator. In this example, selection of the indicator 1304(1) may be reflected in a list 1306. Other embodiments are also possible.

The graphical interface 1300 may represent the various CO₂ capture devices 102, which may be installed within a geographic region. The graphical interface 1300 may be provided to a customer application or to an administrative (control) application to allow a user to view data from the various installations. Other embodiments of a graphical interface and other visualizations of the data are also possible.

The CO₂ source may include, for example, a fermentation tank of a microbrewery. In other embodiments, the CO₂ source may include residential sources, such as water heaters, furnaces, and other sources. In some examples, at least one of the CO₂ capture devices 102, the local server 112 or the control and maintenance server 114 may determine statistics associated with the capture of the CO₂ gas and may provide the data to an application or to a webpage. The location of the CO₂ capture devices 102 may be represented graphically on the map 1302. Other implementations are also possible.

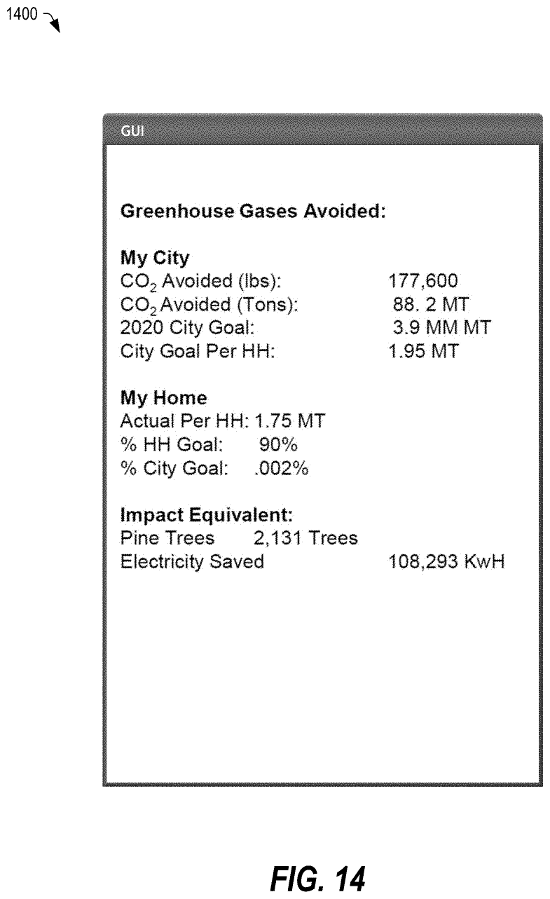

FIG. 14 depicts a graphical interface 1400 accessible by an operator to view data associated with a CO₂ capture device 102, in accordance with certain embodiments of the present disclosure. In this example, the graphical interface 1400 may display the information in a number of ways. For example, the data may be organized and displayed by city or by residence. Further, the data may be presented in terms of the corresponding number of trees.

In the illustrated example, the graphical interface 1400 may include data related to greenhouse gases avoided, organized by city, home, and impact equivalent (in terms of trees, electricity saved, or other impact equivalents. Other embodiments of a graphical interface and other visualizations of the data are also possible.

In conjunction with the systems, methods, and devices described above with respect to FIGS. 1-14, a computing device may include a display, a network interface configured to receive a signal through a network from a carbon dioxide (CO₂) capture device configured to separate CO₂ from a mixed gas waste stream, and a processor coupled to the network interface and the display. The processor may be configured to provide a graphical interface to the display. The graphical interface may include one or more selectable options and may include data related to at least one of the CO₂ capture device and production of purified CO₂ gas from the CO₂ capture device. The computing device may include a smartphone, a tablet computer, a laptop computer, another computing device, or any combination thereof.

In some implementations, a real-time network of hardware, software, and sensors cooperate to enable remote monitoring, analyzing, measuring, communicating, impacting, and controlling a CO₂ gas capture process. The system may include a piece of remote field equipment configured to receive a waste gas stream and to capture CO₂ gas. The system may further include sensors and circuitry configured to capture data corresponding to the capturing of the CO₂ gas and software configured to receive a set of chemical process input parameters and a set of desired chemical process output parameters. In some implementations, a software interface may be accessible by an operator to control a set of chemical process control parameters to achieve selected chemical process output parameters.

Further, the system may include remote monitoring and analysis software that may receive the data from the sensors and that may perform data analytics. In some embodiments, the software may include an open Application Programming Interface (API), which may allow customers to import data to a website to track the CO₂ impact in real-time. Other implementations are also possible.

In some embodiments, the system may be configured to allow any piece of remote field equipment for performing complex chemical processing to be monitored, controlled, and operated remotely, using a portable computing device. Further, in some embodiments, multiple installations situated around the world may be controlled primarily through a single interface provided in a central control center or provided to a portable computing device. Other implementations are also possible.

Although the present invention has been described with reference to preferred embodiments, workers skilled in the art will recognize that changes may be made in form and detail without departing from the scope of the invention.

What is claimed is:

1. A computing device comprising:
a display;
a network interface to couple to a network to receive a signal from a carbon dioxide (CO₂) capture device configured to separate CO₂ from a mixed gas waste stream; and
a processor coupled to the network interface and the display, the processor to:
determine data related to operation of the CO₂ capture device; and
provide a graphical interface to the display, the graphical interface including one or more selectable controls and including the determined data, the determined data including one or more of first data corresponding to one or more parameters of the CO₂ capture device or second data corresponding to production of purified CO₂ gas from the CO₂ capture device.

2. The computing device of claim 1, wherein the computing device comprises a smartphone.

3. The computing device of claim 1, wherein the display comprises a touchscreen display accessible by the operator to interact with the one or more selectable options to control one or more parameters of the CO₂ capture device.

4. The computing device of claim 1, wherein the one or more selectable options are accessible by an operator to selectively adjust an inlet flow rate of the mixed gas waste stream.

5. The computing device of claim 1, wherein the one or more selectable options are accessible by an operator to selectively adjust at least one of a temperature and an operating pressure of at least one component of the CO₂ capture device.

6. The computing device of claim 1, wherein the graphical interface includes:
a set of input parameters corresponding to sensor signals of the CO₂ capture device corresponding to separation-process parameters of an input stream of the CO₂ capture device;
a set of output parameters corresponding to production of a purified CO₂ output stream of the CO₂ capture device; and
the one or more selectable options including at least one selectable option accessible by an operator to control at least one output parameter of the set of output parameters of the CO₂ capture device to adjust a selected chemical process output of the CO₂ capture device.

7. The computing device of claim 6, wherein, in response to selection of the at least one selectable option, the processor is configured to transmit a control signal to the CO₂ capture device to adjust operation of at least one controllable element of the CO₂ capture device.

8. The computing device of claim 7, wherein the control signal is sent through a first communications link between the network interface and a server and from the server to the CO₂ capture device the CO₂ capture device.

9. A system comprising:
a computing device including:
a touchscreen display configured to present images and data and to receive operator selections;
a network interface configured to receive a signal through a network from a carbon dioxide (CO₂) capture device configured to separate CO₂ from a mixed gas waste stream; and
a processor coupled to the network interface and the touchscreen display, the processor configured to provide a graphical interface to the touchscreen display, the graphical interface including one or more selectable options and including data related to controllable parameters of the CO₂ capture device.

10. The system of claim 9, further comprising the CO₂ capture device coupled to a waste gas stream source and configured to produce an output gas stream including the purified CO₂ gas.

11. The system of claim 9, wherein the graphical interface includes:
a set of input parameters corresponding to sensor signals of the CO₂ capture device corresponding to separation-process parameters of an input stream of the CO₂ capture device;
a set of output parameters corresponding to production of a purified CO₂ output stream of the CO₂ capture device; and
the one or more selectable options including at least one selectable option accessible by an operator to control at least one output parameter of the set of output parameters of the CO₂ capture device to adjust a selected chemical process output of the CO₂ capture device.

12. The system of claim 11, wherein, in response to selection of the at least one selectable option, the processor is configured to transmit a control signal to the CO₂ capture device to adjust operation of at least one controllable element of the CO₂ capture device.

13. The system of claim 12, wherein the control signal is sent through a first communications link between the network interface and a server and from the server to the CO₂ capture device the CO₂ capture device.

14. The system of claim 9, wherein the one or more selectable options are accessible by an operator to selectively adjust an inlet flow rate of the mixed gas waste stream.

15. The system of claim 9, wherein the one or more selectable options are accessible by an operator to selectively adjust at least one of a temperature and an operating pressure of at least one component of the CO₂ capture device.

16. A computing device comprising:
a touchscreen display;
a network interface configured to receive a signal through a network from a carbon dioxide (CO₂) capture device coupled to a waste gas stream source and configured to separate CO₂ gas from a mixed gas waste stream to produce an output gas stream including a purified CO₂ gas; and
a processor coupled to the network interface and the touchscreen display, the processor configured to provide a graphical interface to the touchscreen display, the graphical interface including data related to controllable parameters the CO₂ capture device and including one or more selectable options accessible by an operator to control operation of the CO₂ capture device.

17. The computing device of claim 16, wherein the graphical interface includes:
a set of input parameters corresponding to sensor signals of the CO₂ capture device corresponding to separation-process parameters of an input stream of the CO₂ capture device;
a set of output parameters corresponding to production of a purified CO₂ output stream of the CO₂ capture device; and
the one or more selectable options including at least one selectable option accessible by an operator to control at least one output parameter of the set of output parameters of the CO₂ capture device to adjust a selected chemical process output of the CO₂ capture device.

18. The computing device of claim 16, wherein, in response to selection of the at least one selectable option, the processor is configured to transmit a control signal to the CO₂ capture device to selectively adjust an inlet flow rate of the mixed gas waste stream.

19. The computing device of claim 16, wherein, in response to selection of the at least one selectable option, the processor is configured to transmit a control signal to the CO₂ capture device to selectively adjust at least one of a temperature and an operating pressure of at least one component of the CO₂ capture device.

20. The computing device of claim 16, wherein the computing device comprises a smartphone.
